(19) United States
(12) Patent Application Publication
(10) Pub. No.: US 2020/0233875 A1
(43) Pub. Date: Jul. 23, 2020

(54) METHODS AND SYSTEMS FOR NUTRITIONAL ANALYSIS

(71) Applicant: EDAMAM, LLC, NEW YORK, NY (US)

(72) Inventors: VICTOR VALENTINOV PENEV, NEW YORK, NY (US); DINKO TENEV, SOFIA (BG); IANKO IGNATIEV, SOFIA (BG)

(73) Assignee: EDAMAM, LLC, NEW YORK, NY (US)

(21) Appl. No.: 16/250,071

(22) Filed: Jan. 17, 2019

(51) Int. Cl. G06F 16/25; G16H 20/60; G06F 17/27

(52) U.S. Cl. CPC G06F 16/258; G06F 17/278; G16H 20/60

(57) ABSTRACT

Methods and systems for nutritional analysis involving receipt, structuring and information extraction unstructured, natural language food related data such as, without limitation, an ingredient, a recipe, a food or a combination of the forgoing to produce in real time, human accuracy level nutrition analysis and diet/allergen tagging of the food related data. The process involves structuring data by mapping it to a database of foods, ingredients, measures, techniques and food qualifiers, combining the mapped data into constructs according to a data model organized in a food ontology, parsing the constructs into meaningful combinations of food, quantities, measures and techniques; and assigning nutrient data to such combinations Further processing involves adjusting nutrient content, or assigning diet information and allergen information to a list of parsed foods or to a recipe, resulting in final data which may then be exported or displayed to a user.

### CROSS-REFERENCE TO RELATED APPLICATIONS

This application claims the benefit of, and is related to, Applicant's following provisional patent application: U.S. Provisional Patent Application No. 62/520,310 titled “METHODS, COMPUTER PROGRAM PRODUCT & SYSTEMS FOR NUTRITIONAL ANALYSIS” filed Jun. 15, 2017, which is incorporated herein in its entirety.

### FIELD OF THE INVENTION

The present invention is generally related to computer implemented methods, systems, and computer program product directed to the nutritional analysis of food, recipes, ingredient lists and the like.

### BACKGROUND OF THE INVENTION

Restaurants, Catering Companies, Food Manufacturers and Recipe Creators, as well as Dietitians, Nutritionists, Health Coaches, Wellness Programs and Population Health Management organizations all have a need to analyze the nutrition of recipes, meals or ingredient lists of meals they develop, prepare, serve, deliver or meal eaten by their customers, patients or employees. Currently, existing options for analyses tend to be very slow and expensive. Many companies also have to analyze hundreds of thousands if not millions of meals in real time, while working within the limitations of a budget. As a result, many companies do not perform the needed analysis, or in cases where they do, settle with less accurate information compiled from free resources available on the Internet. There is a real trade-off between price and accuracy of the analysis. As such, there is a need for an efficient, accurate and affordable system and method of performing nutritional analysis of food, recipes, ingredient lists and the like.

### SUMMARY OF THE INVENTION

An aspect of an embodiment of the present invention contemplates systems, methods, computer program product and non-transitory computer readable device(s) which are directed to the implementation and operational functionality of, inter alia, the provision of highly accurate nutritional analysis of recipes, ingredients and the like in real time at a fraction of the cost for similar analyses.

An aspect of an embodiment of the present invention contemplates systems, methods, computer program product and non-transitory computer readable device(s) which are directed to enabling a user to enter, in a natural language, any recipe or ingredient list and analyze it with one click or tap, thereby obtaining highly accurate and detailed nutrition analysis in a short period of time. Aspects of embodiments of the present invention allow for use of true natural language, the way one would describe such a recipe or ingredient list to a friend or a health coach. It also returns information for up to 70 nutrients and automated calculation of the applicability of the analyzed recipe or ingredient list for 40+ most popular allergen conditions or diets (e.g., paleo, gluten-free, vegan, etc.) Aspects of embodiments of the present invention enable the ingestion of unstructured, free, natural language text of foods, recipes or ingredient lists and produce in real-time nutritional analysis and diet/allergen tags with human level accuracy.

The invention is driven by the need to allow human level accuracy in nutrition analysis of free text recipes or ingredient lists. To that extent, aspects of embodiments of the present invention using systems, methods, and computer program product, takes true natural language in the food domain and converts such natural language into structured and quantifiable nutrition data. There are two primary uses of the invention: consumer and businesses. Consumers, as well as individual restauranteurs or dietitians can type in a natural language, copy/paste or speak into a device using voice recognition, the recipe or ingredient list they want analyzed. Then, systems, methods, and computer program product, in aspects of embodiments of the present invention, would take the entered data and break it into its component parts. This may be done using technology specifically focused on the food domain. Systems, methods, and computer program product according to aspects of embodiments of the present invention, produce from the text, a set of structured data elements, such as ingredients, quantities, measure and cooking techniques. For instance, the phrase “one bunch of kale” may be broken down using, in aspect(s) of embodiment(s) of the present invention, systems, methods, and computer program product thereby recognizing the word “bunch” as a measure of the food article (i.e. “kale”) and assigning weight to it, while the word “one” is recognized as the quantity of the food article. Similarly, the additional exemplary phrase “1 cup minced onion” may be broken down using, thereby enabling recognition of the word “minced” as a technique that creates different density of the food used and its weight different and distinct than, say, “one cup chopped onion”, “salt and pepper to taste” (applying quantities to both ingredients based on software code and/or computer program product that take into account all other ingredients in the list or recipe), “3 rib-eye steaks” (understanding what is the most common size and fat content of a rib-eye steak), etc. Once the recipe is parsed, it is matched against a dataset of nutrient content, contained within a database according to an aspect of an embodiment of the present invention, for the recognized foods and an initial calculation of the nutrition analysis is done using simple formulas. Then, the analysis proceeds to the next stage, whereby cooking techniques which impact the nutrient content are considered. This stage (post-processing) may use a de facto set of proprietary software code, which may be used to calculate, for example, how much fat gets absorbed into food when it is fried, how much of a marinade clings on to food, what happens if you bake in salt or if you make a stock and throw away the solids, etc. At this time, a different set of software code may be applied to calculate the applicability of the recipe or ingredient list to all allergen conditions or popular diets (e.g., low-sodium, soy-free, vegetarian, etc.) In one aspect of an embodiment of the present invention, the full analysis, including parsing, matching, and post-processing takes on average 400 milliseconds. The final result is then displayed to user.

A similar process may be used for business users, but instead of having an actual graphic interface to enter the recipe or ingredient list and receive the final analysis, an Application Programming Interface (API) may be used. The business user may submit the ingredient and/or recipe information using machine readable format (in a natural language) and subsequently receive the results in machine readable format.

Aspects of embodiments of the present invention may be implemented as: (a) a consumer application either on the web or as a native mobile application; (b) as an Application Programming Interface (API) to be used by third parties, who can develop user interfaces on top of it; or (c) as an integrated module in a larger software solution that does diet tracking (health and wellness companies) or recipe and inventory management (restaurants and catering businesses).

Because of the food specific natural language understanding and the proprietary systems, processes, methods, software code, computer program product and/or apparatuses used in post-processing, the invention is essentially able to provide real-time, highly accurate nutrition analysis of a recipe or ingredient list, which no other currently available solution can. Its advantage is the ability to significantly reduce a user's transaction cost in terms of time and money.

An aspect of an embodiment of the present invention contemplates a computer-implemented method of nutritional analysis which takes unstructured food-related text and turns it into a structured data set with nutrition and diet/allergen information virtually in real time, where the process includes, the following execution steps on one or more processors: enabling receipt of one or more collections of unstructured food related data, where the collection(s) of unstructured food related data may be any one of one or more of: an ingredient, a recipe, a food, a meal, a measure, a qualifier, a technique, or a combination of the forgoing, extracting and identifying the one or more collections of unstructured food related data using a predetermined process, where extraction and identification of the one or more collections of unstructured data results in one or more structured data sets, mapping the structured data set(s) to an entity class, combining the extracted the structured data set(s) into one or more constructs, combining the construct(s) into meaningful combinations of food, quantities, measures and techniques, where the combination results in one or more parsed combinations, assigning nutrient content, diet information and allergen information to the parsed combination(s) resulting in final data, where the assignment is done using a database of nutrient content, diet information and allergen information for a wide range of foods, and exporting the final data. In an aspect of an embodiment of the present invention, each step takes place by execution of specific sets of machine codes on processor(s) configured to perform a predefined set of basic operations in response to receiving a corresponding basic instruction selected from a predefined native instruction set of codes, where the specific sets of machine codes are selected from the native instruction set.

In an aspect of an embodiment of the present invention, the computer implemented method may include the step of applying a series of steps to make at least one adjustment to the structured data set(s).

In an aspect of an embodiment of the present invention, the computer implemented method may include the additional steps of: checking for compatibility between the one or more constructs and ensuring that correct quantities and measures are applied to the structured data set(s).

In an aspect of an embodiment of the present invention, the computer implemented method may include the step of adjusting sodium amount in the structured data set(s).

In an aspect of an embodiment of the present invention, the computer implemented method may include the step of calculating the amount of fat absorption based on a frying technique parsed in the structured data set(s).

In an aspect of an embodiment of the present invention, the computer implemented method may include any one or more steps of: calculating amount of nutrients in the final data, calculating recommended daily intake (RDI) for each nutrient, tagging the final data for a range of predefined diets.

Another aspect of an embodiment of the present invention contemplates a system for nutritional analysis which takes unstructured food-related text and turns it into a structured data set with nutrition and diet/allergen information virtually in real time, where the system may include: one or more input devices, one or more of: a search instances module, an API instances module, a scraper module configured to search and obtain information from the internet, where any one or more of the input device(s), search instances module, API instances module, and scraper module are configured for receipt of one or more collections of unstructured food related data, where the collection(s) of unstructured food related data is any one or more of: an ingredient, a recipe, a food, a meal, a measure, a qualifier, a technique, and/or a combination of the forgoing. The system may also include an extractor module, where the extractor module receives the collection(s) of unstructured food related data from the scraper module and where any one or more of: the search instances module, API instances module, the extractor module operates upon the collection(s) of unstructured food related data, and one or more database(s) in communication with the extractor module. In an aspect of an embodiment of the present invention, the database(s) may be in communication with any one or more of the search instances module or API instances module.

In another aspect of an embodiment of the present invention, the system may include an elastic search cluster module in communication with any one or more of: the search instances module, the API instances module, the extractor module and the at least one database.

In another aspect of an embodiment of the present invention, the system may include a nutrition wizard interface module in communication with any one or more of: API instances module, extractor module, elastic search cluster modules, search instances module.

In another aspect of an embodiment of the present invention, the system may include an entity loader module in communication with any one or more of: the search instances module, the API instances module, the extractor module.

A further aspect of an embodiment of the present invention contemplates computer program product with a non-transitory computer readable medium having interfaces stored on it for causing a processor-based control logic to conduct nutritional analysis which takes unstructured food-related text and turns it into a structured data set with nutrition and diet/allergen information virtually in real time, which may involve executing control logic on at least one processor, thereby implementing the steps of: enabling receipt of one or more collections of unstructured food related data, where the collection(s) of unstructured food related data may be any one of one or more of: an ingredient, a recipe, a food, a meal, a measure, a qualifier, a technique, or a combination of the forgoing, extracting and identifying the one or more collections of unstructured food related data using a predetermined process, where extraction and identification of the one or more collections of unstructured data results in one or more structured data sets, mapping the structured data set(s) to an entity class, combining the extracted the structured data set(s) into one or more constructs, combining the construct(s) into meaningful combinations of food, quantities, measures and techniques, where the combination results in one or more parsed combinations, assigning nutrient content, diet information and allergen information to the parsed combination(s) resulting in final data, where the assignment is done using a database of nutrient content, diet information and allergen information for a wide range of foods, and exporting the final data.

In a further aspect of an embodiment of the present invention, the computer program product may include control logic for applying a series of steps to make at least one adjustment to the structured data set(s).

In a further aspect of an embodiment of the present invention, the computer program product may include control logic for checking for compatibility between the one or more constructs and ensuring that correct quantities and measures are applied to the structured data set(s).

In a further aspect of an embodiment of the present invention, the computer program product may include control logic for adjusting sodium amount in the structured data set(s).

In a further aspect of an embodiment of the present invention, the computer program product may include control logic for calculating fat absorption amount based on a frying technique parsed in the structured data set(s).

In a further aspect of an embodiment of the present invention, the computer program product may include computer readable code for any one or more of: calculating amount of nutrients in the final data, calculating recommended daily intake (RDI) for each nutrient, tagging the final data for a range of predefined diets.

A yet further aspect of an embodiment of the present invention contemplates a non-transitory computer readable device having control logic stored on the device for causing a computer-based interface to implement nutritional analysis which takes unstructured food-related text and turns it into a structured data set with nutrition and diet/allergen information virtually in real time, where the control logic may include computer readable program code for: enabling receipt of one or more collections of unstructured food related data, where the collection(s) of unstructured food related data may be any one of one or more of: an ingredient, a recipe, a food, a meal, a measure, a qualifier, a technique, or a combination of the forgoing, extracting and identifying the one or more collections of unstructured food related data using a predetermined process, where extraction and identification of the one or more collections of unstructured data results in one or more structured data sets, mapping the structured data set(s) to an entity class, combining the extracted the structured data set(s) into one or more constructs, combining the construct(s) into meaningful combinations of food, quantities, measures and techniques, where the combination results in one or more parsed combinations, assigning nutrient content, diet information and allergen information to the parsed combination(s) resulting in final data, where the assignment is done using a database of nutrient content, diet information and allergen information for a wide range of foods, and exporting the final data.

In a yet further aspect of an embodiment of the present invention, the non-transitory computer readable device may include computer readable code for applying a series of steps to make at least one adjustment to the structured data set(s).

In a yet further aspect of an embodiment of the present invention, the non-transitory computer readable device may include computer readable code for checking for compatibility between the one or more constructs and ensuring that correct quantities and measures are applied to the structured data set(s).

In a yet further aspect of an embodiment of the present invention, the non-transitory computer readable device may include computer readable code for adjusting sodium amount in the structured data set(s).

In a yet further aspect of an embodiment of the present invention, the non-transitory computer readable device may include computer readable code for calculating fat absorption amount based on a frying technique parsed in the structured data set(s).

In a yet further aspect of an embodiment of the present invention, the non-transitory computer readable device may include computer readable code for any one or more of: calculating amount of nutrients in the final data, calculating recommended daily intake (RDI) for each nutrient, tagging the final data for a range of predefined diets.

### BRIEF DESCRIPTION OF THE DRAWINGS

FIG. 1 illustrates a system utilized by a process according to an aspect of an embodiment of the present invention.

FIG. 2 illustrates an exemplary input device with recipe/ingredient input content according to an aspect of an embodiment of the present invention.

FIG. 3 illustrates a process/system component diagram according to an aspect of an embodiment of the present invention.

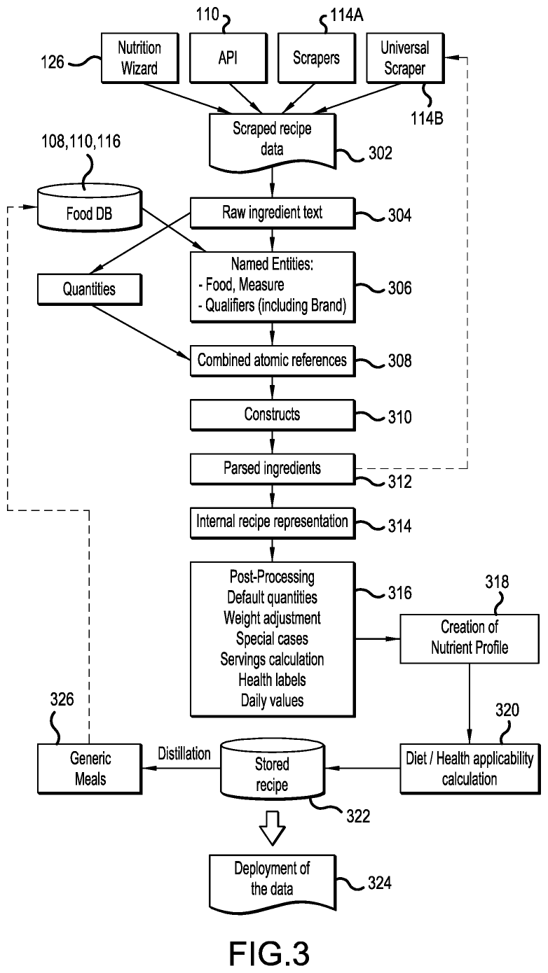

FIG. 4 illustrates an exemplary display of a resultant analysis according to an aspect of an embodiment of the present invention.

FIG. 5 illustrates a user submitting information to a system via voice input according to an aspect of an embodiment of the present invention.

### FURTHER DESCRIPTION OF THE INVENTION

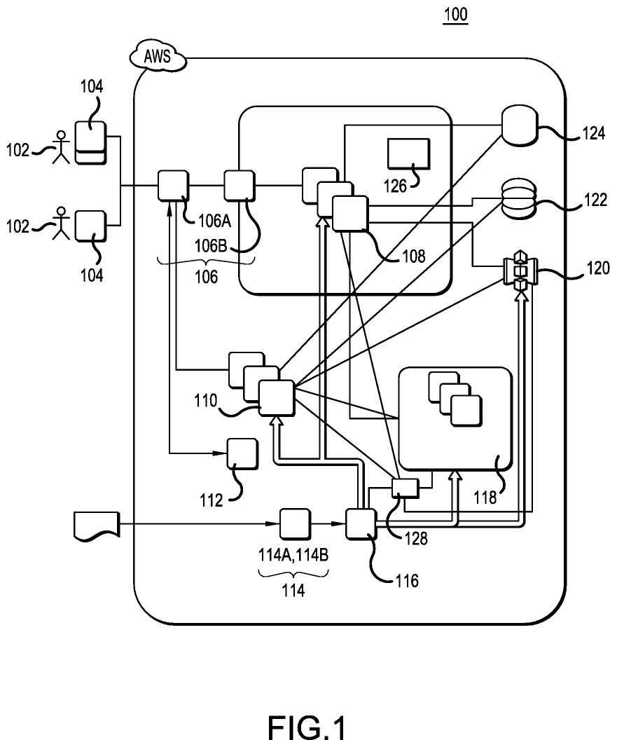

Referring now to FIG. 1, a system 100 is shown according to an aspect of an embodiment of the present invention. System diagram 100 shows user 102 which could be an individual seeking the nutritional analysis of a recipe, ingredient or the like. User 102 may use input device 104 such as a mobile device to input the recipe, ingredient etc. into system 100. System 100 may be characterized by load balancing module 106—which may include, in an aspect of an embodiment of the present invention, first portion 106A tasked with receiving incoming data, and second portion 106B which processes and determines how the incoming data is balanced amongst system 100's modules. In an aspect of an embodiment of the present invention, load balancing module 106 may comprise of one portion performing both functions. Load balancing module 106 may operate by certain rules—e.g. if there are ten incoming messages, load balancing module 106 may be able to assign certain numbers to different machines. In another aspect of an embodiment of the resent invention, load balancing module 106 may determine and send information from a particular source, which had previously used system 100, to the server or component of system 100 which was previously assigned to the source.

System 100 may also include Search Instances module 108 and API Instances Module 110, both connected in an aspect of an embodiment of the present invention, to load balancing module 106. API instances module 110 collects data from other machines or components of system 100 and converts the collected data into an applicable format. In one aspect of an embodiment of the present invention, this may include the JSON format. In another aspect of an embodiment of the present invention, API instances module 110 may include two or more modules. Display and output of system 100 may be determined by API instances 110. Food, recipes, meals, ingredient lists, combinations of the foregoing and the like may be submitted by customers—individuals or companies and ingested via search instances module 108. In an aspect of an embodiment of the present invention, search instances module 108 may include two or more modules.

Directly connected to device 104 is scraper module 114. Scraper module 114 may include code that enables scraper module 114 to collect data, including food, recipes, meals, ingredient lists and the like from the internet. In an aspect of an embodiment of the present invention, scraper module 114 may include code that enables scraper module 114 to search specific websites, or be universal in its data collection with no preference with respect to the type of website. Scraper module 114, in an aspect of an embodiment of the present invention, collect or receive the scraped data e.g. food, recipes, meals, ingredient lists and the like line by line along with lists, instructions, techniques, quantities in a row format and taken as text.

Connected to scraper module 114 is extractor module 116 which performs extraction of nutritional analysis from the data obtained by scraper module 114. Extractor module 116 also performs analyses on the extracted data. Extractor module, in a preferred aspect of an embodiment of the present invention, may include two components:

Information Extractor:

This, in a preferred aspect of an embodiment of the present invention, is a component which allows for food/nutrition specific information extraction from a recipe, ingredient, food or combination thereof. The basis of the technology is the ability to recognize and structure free-flowing natural text, breaking it into data relevant components, such as ingredients, quantities and measures. In an aspect of an embodiment of the present invention, this component is resident on components of system 100 which conduct extraction, namely, search instances module 108, API instances module 110 and extractor 116.

Natural Language Processor:

This, according to an aspect of an embodiment of the present invention, is another component of each of search instances module 108, API instances module 110 and extractor module 116. The work of search instances module 108, API instances module 110 and extractor module 116 is based on proprietary set of rules and code to process natural language as it relates to food. In the process, system 100, in one aspect, has a very extensive and cultural/language specific database which may be used to understand how humans think and speak about food and translate this natural language into a hard, measurable data.

Connected to extractor module 114, API instances module 110 and search instances module 108 is elastic search cluster module 118 which is optimized for search queries made to system 100. Elastic search cluster module 118, in an aspect of an embodiment of the present invention, maintains an index or reference information for each extracted food, recipes, meals, ingredient lists and the like which refers to information regarding food, recipes, meals, ingredient lists and the like that have been extracted by search instances module 108, API instances module 110 and extractor module 116. Extracted information is stored in database 120 in structured RDF format. Each indexed food, recipes, meals, ingredient list etc. is uniquely identified, tagged or indexed search instances module 108, API instances module 110 and extractor module 116 and the indexing/tagging information is saved on elastic search cluster module 118. The reference information or index points to the particular extracted food, recipes, meals, ingredient lists and the like as stored in database 120. This arrangement is advantageous computationally and cost-wise as this is a more efficient way to storing data and not overloading memory requirements of components of system 100, with elastic search cluster 118 in particular. In an aspect of an embodiment of the present invention, during each extraction process, each text component is tagged as technique, quantity, measure etc. and saved in database 120. In another aspect of an embodiment of the present invention, elastic search cluster module 118 may include two or more nodes or sub-modules. In yet another aspect of an embodiment of the present invention, elastic search cluster module 118 may operate to determine what a query is and where the data sought is stored i.e. which one of databases 120-124 has the sought information.

System 100 may also include databases 120, 122 and 124. It should be noted that the number of databases shown is for illustrative purposes only and that fewer or more databases may still be used according to aspects of embodiments of the present invention.

In one aspect of an embodiment of the present invention, database 120 may be an S3 database. In an aspect of an embodiment of the present invention, database 120 may have different responsibilities. Database 120 may collect and store all data collected, extracted and/or received by search instances module 108, API instances module 110, scraper module 114 and extractor module 116. In another aspect of an embodiment of the present invention, database 120 may store all incoming data to system 100. Database 120, in a preferred aspect of an embodiment of the present invention, may have different functionalities as further described below.

Database 122 may, in one aspect of an embodiment of the present invention, be connected to search instances module 108 and API instances module 110. In another aspect of an embodiment of the present invention, database 122 may store information regarding particular users of system 100, including, without limitation, user information, user recipes, user preferences, etc. that will enable system 100 to call on when the same user uses the system again. In an aspect of an embodiment of the present invention, database 122 may be a Dynamo database.

Database 124 may, in an aspect of an embodiment of the present invention, store certain specific user information, including, without limitation, user electronic mail addresses, user sessions, gender, sex, age etc. Database 124 assists in serving particular requests to system 100 for instance a 40-year-old man seeking nutritional analysis of a particular recipe—in which case system 100 would take the individual's age and sex in calculating the individual's nutritional needs. In an aspect of an embodiment of the present invention, database 124 may be a Simple Database.

System 100 may also include nutrition wizard module 126 which is a web-site or mobile application interface to system 100) and all related displays. Nutrition wizard module 126 may be connected to particular components of system 100, including, but not limited to extractor module 116, databases 120-124, elastic search cluster module 118, API instances module 110 and search instances 108. Following conversion of extracted data into preferable format, e.g. JSON format, API instances module 110 may send the extracted information to nutrition wizard module 126 for the extracted information's use or display.

Extraction, in a preferred embodiment, may take place at three components of system 100—at search instances module 108, API instances module 110 and extractor module 116 with each extraction being dependent on how the information is collected and how it is to be displayed, stored etc. For instance, information extracted for later use may be extracted at extraction module 116. Extracted information which is needed for immediate analysis may be extracted at API instances module 110 and stored in database 122 while extracted information which is extracted in response to a search e.g. search conducted by a user searching for a recipe or seeking nutritional analysis of same, may be extracted by search instances module 108.

In an aspect of an embodiment of the present invention, each of API instances module 110, search instances module 108 and elastic search cluster module 118 may have referential data or indexing information pointing to specific data stored in any or all of databases 120-124. In another aspect of an embodiment of the present invention, each of API instances module 110, search instances module 108 and elastic search cluster module 118 may include code for referential data or indexing information for retrieving the top references, instead of an entire database, for nutritional data stored on any or all of databases 120-124. This information may include, but not be limited to, title, recipe, picture, ingredient(s), nutritional data, serving data etc. For instance, when a user makes a search request, search instances module 108 may then, using referential data or indexes stored on it, pull up the top 100 results corresponding or relevant to the user's search request. In another aspect of an embodiment of the present invention, search instances module 108 may refer to elastic search cluster module 118 to use its index to locate the sought information.

In a preferred aspect of an embodiment of the present invention, different components of system 100 may work together in providing results for a search. In one aspect of an embodiment of the present invention, after the extraction of a food, recipes, meals, ingredient lists and the like, by any one of search instances module 108, API instances module 110 or extractor module 116, may request the referential information or index from elastic search cluster module 118 for provision of the nutritional analysis of the extracted food, recipes, meals, ingredient lists etc. Elastic search cluster module 118 would then call up the actual full structured data from database 120, where the extracted information and, in one aspect, nutritional analysis data is stored.

System 100 may include a number of additional or specific components as further described below:

Food Ontology:

This is an ontology developed as an aspect of an embodiment of the present invention. The ontology provides the basic data model for organizing food and nutrition data. It is built on the principles of linked data and triplet logic to create low-level artificial intelligence graph organization of data vs. traditional table form databases. An ontology is a representation of a data model that describes categories and entities in a particular domain and their relationship to each other. For example, in an aspect of an embodiment of the present invention, “Recipe” is an entity and a “food” is an entity. A food can belong to a recipe, which is a relationship between those two concepts and a “recipe” can be a “food” (e.g., “spaghetti bolognese” is a recipe that is food), which is another relation between those two entities. The food ontology may, in one aspect of an embodiment of the present invention, reside on database 120. In another aspect of an embodiment of the present invention, it may also reside on search instances module 108, API instances module 110 and extractor module 116.

Food Knowledge Graph Database:

Built on top of the ontology residing on database 120, the Food Knowledge Graph Database is an actual semantically organized database with 1.5 million recipes, over 17 million ingredient lines, over 550,000 foods/ingredients, over 300,000 food aliases, 70+ nutrients, 40+ diets and a number of other recipe attributes, such as cooking techniques, level of difficulty, associated cooking tools, cuisine, meal type, etc. In an aspect of an embodiment of the present invention, it may reside on search instances module 108, API instances module 110 and extractor module 116.

Knowledge Base Generator:

This, in an aspect of an embodiment of the present invention, is an additional component which enables the population of extracted data into the Food Knowledge Graph Database of search instances module 108, API instances module 110 and extractor module 116 mentioned above.

Entity Loader Module:

In an aspect of an embodiment of the present invention, the entity loader module 128 is a representative of a range of possible software programs that can trigger the post-processing operations as will be discussed in further detail below.

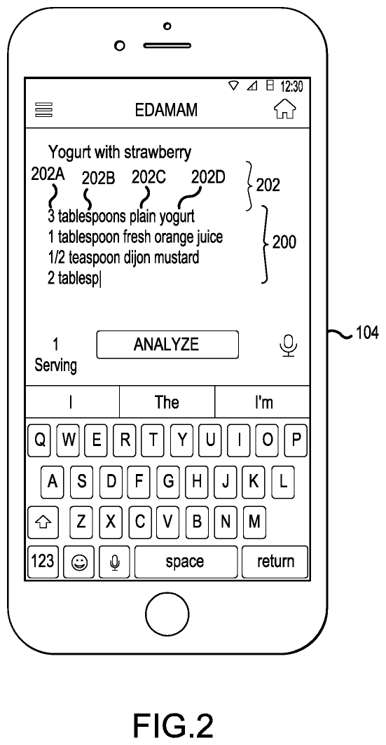

Referring now to FIGS. 2 & 3, an exemplary input telecommunications device 104 with recipe/ingredient input content 200 and a nutrition analysis process/system component diagram 300 are shown according to aspects of embodiments of the present invention. As shown, food, recipes, meals, ingredient lists or a combination of the foregoing as unstructured, free, natural language text may be submitted, using a telecommunications device, which, may have a processor/computer (such as a desktop computer, laptop, notebook, tablet, mobile phone, or the like) may be entered, collected or submitted into system 100 using a variety of methods including, without limitation: input into nutrition wizard module 126 using a web-site interface via a web-browser or a mobile application interface, machine or computer input into API instances module 110 via JSON delivered data through an API, scraper modules 114 (such as scraper module 114A and/or universal scraper module 114B which, in aspects of embodiments of the present invention, “scrape” or obtain text, images and other data from web-pages). In an aspect of an embodiment of the present invention, the unstructured text of the food, recipe, meal, ingredient list (or a combination of the foregoing) may be sent in another format, such as Word or Excel and converted then to a format which can be processed by the API instances module 110. The nutrition wizard module 126 may also support data or information submission via voice, using as an intermediary external voice recognition program such as Google Now, Amazon Alexa or Apple Siri.

In all cases the recipe or ingredient list 200 may be submitted “as is” in a natural language, easily understood by a human, but not structured or readable by a machine that can analyze it or do anything with it. There is no need to write ingredients in a specific format or use drop down menus to identify the right quantity of an ingredient. An important element of the invention is that recipes, ingredient lists or foods are submitted in a natural language, the way a human may describe them to another human, not to a machine.

At step 302, recipes scraped from the web may be ingested using scraper module 114A (developed for specific web-sites) or Universal Scraper module 114B (which works on most websites). Recipes submitted by customers—individuals or companies—are ingested via the API Instances module 110. In an aspect of an embodiment of the present invention, the raw recipe data is stored in a document format i) for scraped recipes from the web, into database 120 and ii) for recipes from an identifiable source, such as dietitian, a restaurant or anyone with a user account who chooses to save analyzed recipe, into the database 122.

Initial Data Structuring:

Once the recipe is ingested in step 302, it is then broken into its main component parts or atomic references 202A-D by the relevant extracting component of system 100 (depending on how the information was ingested) in step 304 as exemplarily outlined below:
title
summary
preparation
ingredient lines
URL (if applicable)
Photo or photo URL (if applicable)

The above recipe data is stored in the same databases as the full recipes from the ingestion step above—in databases 120 and 122.

Extraction:

Next, in step 306, the information from step 304 is further analyzed using natural language processing technology to identify entities within the information that have relevance to analyzing it nutritionally and labeling it for appropriateness to common diets, health or allergen conditions. The involved components of process 300 is search instances module 108, API instances module 110 and extractor module 116, with the activity depending on how the original unstructured text of the food, recipe, meal, ingredient list (or a combination of the foregoing) was ingested or received. For example, in an aspect of an embodiment of the present invention, if the unstructured text is received from another machine or computer, the relevant extracting module would be API instances 110. If, on the other hand, the unstructured text is received as a result of an input by an end-user via the Nutrition Wizard module (126), then either the API instances (110) or the search instances module 108 would perform its extraction. Finally, if the unstructured text is obtained by scraper 114, then extractor module 116 would perform its extraction. The extraction works as follows:
The relevant extracting module first identifies quantities, which are numbers.
Next, the relevant extracting module identifies named entities as outlined below:
   a. Foods, which are the common foods referring to gazetteer with the name of most foods used in a natural language
   b. Measures, such as pound, cup, bunch, etc.
   c. qualifiers e.g., “green” in “green apple,”
   d. techniques, to define the proper ingredient and measure e.g., to calculate the weight of “one cup minced onion” or “one cup chopped onion”

All the named entities are identified using a predetermined process and mapped to an entity class, as defined by system 100's internally developed data model which is a semantically organized database of the food universe, stored within database 120 of the system. In an aspect of an embodiment of the present invention, this may also be stored on any one or more of search instances module 108, API instances module 110 and extractor module 116. Aspects of embodiments of the present invention contemplate extension of search instances module 108, API instances module 110 and extractor module 116 to enable definition of new entities in the Ontology which can then identified by search instances module 108, API instances module 110 and extractor module 116. Examples may include geography, location, chemical composition, manufacturers, brand, etc.

In an aspect of an embodiment of the present invention, the predetermined process involves matching the unstructured text of the food, recipe, meal, ingredient list (or a combination of the foregoing) against name(s) of entities in the ontology to identify them as such entities. For example, if in the ontology, the entity “food” has “rice” as a particular representation of a “food”, if “rice” is found by the relevant extractor module in the text, it will be marked as potentially indicating a “food.”

For every class of entities, the contemplated invention has developed a very comprehensive and constantly updated database of food names, measures, quantities, cooking techniques and qualifiers, as well as any other relevant data, such as allergen information. For example, this database contains the word “rice” as a representative of entity type “food” from the “food” entity class.

In the process diagram, this database is identified as Food DB. It is stored locally on every machine that performs extraction and presents nutrition data, using the contemplated process, namely, search instances module 108, API instances module 110 and extractor module 116.

Construct Creation:

Next, in steps 308 and 310, the extracted entities are combined, by the extracting module, into “Constructs” as defined by an aspect of an embodiment of the present invention. These constructs represent Natural Language Understanding in the food domain.

Some construct examples, include, but are not limited to, the following
“3 cups” is a construct combining the quantity “3” with the measure “cup”
“cooked rice” is a construct combining the food “rice” with the technique “cooked”
“3 cups cooked rice” is a construct of constructs, allowing the system to assign specific weight to a specific ingredient prepared in a specific way
Additional constructs deal with elements of the language, where a choice needs to be made—e.g. (i) “Aubergine or squash” is a construct with a particular feature, which indicates that one of the two foods in an ingredient line needs to be chosen or (ii) “salt to taste” is a construct to indicate an ingredient where specific quantity is not provided and an additional code needs to be executed for analysis of this construct (discussed further below).

All “Constructs” are additive structures developed by an aspect of an embodiment of the present invention with specific focus on the food domain and food domain knowledge in order to create natural language understanding.

In an aspect of an embodiment of the present invention, should system 100 have an unknown food, recipe, meal, ingredient list (or a combination of the foregoing) that is not stored on database 120, loaded or presented, the extracting module, through execution of code, will then take the unknown food, recipe, meal, ingredient list (or a combination of the foregoing) analyze it ingredient by ingredient and query database 120 about what is known (i.e. stored in database 120) regarding each ingredient. Each measure, quantity, qualifiers (e.g. brand) is identified and combined as atomic references into constructs at which point the relevant extracting module would query database 120 regarding what is known regarding these constructs.

Parsing:

Next in step 312, all constructs are reassembled, by the relevant extracting module into meaningful combinations of food, quantities, measures and techniques that produce ingredient lines to which a specific amount (weight) of a specific food (ingredient) can be assigned. The thus reassembled ingredient lines carry enough information to be able to assign nutrient values and diet/allergen attributes. The result is “parsed” ingredient. In another aspect of an embodiment of the present invention, the reassembled ingredient lines are reassembled into a recipe, presented in RDF format and indexed within database 120 to enable its searchability.

An aspect of an embodiment of the present invention enables the relevant extracting module to assign nutrient content, using a very deep database of nutrient content for a wide range of foods for standard quantities.

Ambiguity Reduction:

During the parsing, the relevant extracting module with its execution of relevant code, checks for compatibility between the constructs within an ingredient line. The relevant extracting module, with its execution of relevant code, may also check to see whether the correct quantities and measures are applied to foods. For example, the process will check if “can” is a valid measure for the food “tomato” and if so apply it. Another example is if 16 fl. oz. is used as a measure for tomatoes—which the system would determine does not make sense as tomatoes are measured in regular ounces, not fluid ounces.

The ambiguity in this example arises from the fact that the word “can” is both a verb and a noun and if used as a noun it may not be relevant contextually to the food in the ingredient line where it is used. In this example, “a can of chopped tomatoes” is a valid ingredient line, but “you can use tomatoes instead” or “a can of sirloin steak” would not be. Execution of the ambiguity reduction code enables use a number of techniques to identify the morphology of words and their relevant proximity to each other in the ingredient line to identify only relevant ingredient data, removing false positives or lines of text that are not ingredient lines.

Internal Recipe Representation:

Steps 310 and 312. i.e. Construct Creation, Parsing and Ambiguity Reduction are essence of the transformation of a free text recipe or ingredient list into a structured data representation (“structured data set”) of the food, recipe, meal, ingredient list (or a combination of the foregoing). The contemplated invention defines this representation as the Internal Recipe Representation. The thus structured data set is stored temporarily “in memory” in step 314 on the machine or module on which the extraction process is performed. It is stored long-term in the database module 120.

Post Processing:

Following completion of step 314, entity loader module 128 activates or triggers a range of post processing code, which adjust or further analyze nutrient content or assign diet or allergen applicability not to a particular ingredient, but to the entire recipe. This is an additional level of analysis in the context of the entire recipe. Examples of the post-processing executable code which could be triggered by entity loader module 128 include:
Salt code—which enables entity loader module 128, upon its execution of this code, to adjust the amount of sodium in a recipe if non-clearly defined amount of salt is used, e.g. “salt and pepper to taste”.
Frying (oil absorption) code—based on “frying” technique parsed in the text and the context of the ingredients, such as “oil” or “butter,” this code enables, upon its execution by entity loader module 128, a formula to be applied to calculate the amount of fat absorbed into the food during the frying process, recognizing that not all fat in the recipe is consumed.
Marinating code—this code enables, upon its execution by entity loader module 128, calculation of the amount of marinade that remains on the food during the cooking process.
Stocks and broths code—this code, upon its execution by entity loader module 128, considers that solids are thrown out in preparation of stocks and broths.
Baking in salt code—this code enables, upon its execution by entity loader module 128, calculation of the amount of sodium retained by the food baked in salt.
Evaporation of water code—this code enables, upon its execution by entity loader module 128, calculation of how much water is lost during cooking, based on the food cooked and the cooking technique applied.
Juicing code—this code enables, upon its execution by entity loader module 128, calculation of the retained and lost nutrients when juice is extracted from foods.

When these codes are executed by entity loader module 128, the relevant extracting module (any one of search instances module 108, API instances module 110 or extractor module 116) is prompted to take process 300 to step 318.

Creation of Nutrient Profile:

Once all ingredients are parsed and the post-processing code executed and applied as needed, any one or more of search instances module 108, API instances 110 and/or extractor module 116, in step 318, calculates the amount of nutrients in the recipe. These include energy (calories), macronutrients (carbs, fat, protein), micronutrients (minerals, vitamins), cholesterol, sodium, sugar, fiber and even individual molecules, such as amino acids and fatty acids. At this stage, any one or more of search instances module 108, API instances 110 and/or extractor module 116 with its execution of relevant code, calculates the recommended daily intake (RDI) for each nutrient, based on specific weight, height, gender, age and activity level of an individual, if provided.

Diet/Health Applicability Calculation:

Once the nutrient profile of a recipe is calculated, a further set of code is executed by any one or more of search instances module 108, API instances 110 and/or extractor module 116 in step 320 to tag the recipe for a range of predefined diets. Examples are “low sodium,” “low sugar,” “high fiber,” “high protein,” etc. Tagging may be done based on the nutrient profile and/or other descriptors found in the data set. At this stage, based on individual food data tagging, any one or more of search instances module 108, API instances 110 and/or extractor module 116 calculates applicability of the recipe for various diets, which are based on inclusion or exclusion of certain foods, rather than particular levels of nutrient intake. Examples are all allergens (diary, soy, gluten, etc.) as well as diets such as “vegan,” “paleo,” “kosher.”

Stored Recipes:

Once all nutrition calculations are per performed the resulting data is represented in step 322 in an RDF format or Resource Description Framework format. This format is the standard for representing semantically organized data. As the contemplated invention's data model is defined by its Food Ontology, all data is eventually represented in RDF and stored in a graph database (not a standard SQL on NoSQL database). Physically, in an aspect of an embodiment of the present invention, the RDF represented recipes may be stored in an S3 database machine (120).

Data Export/Deployment:

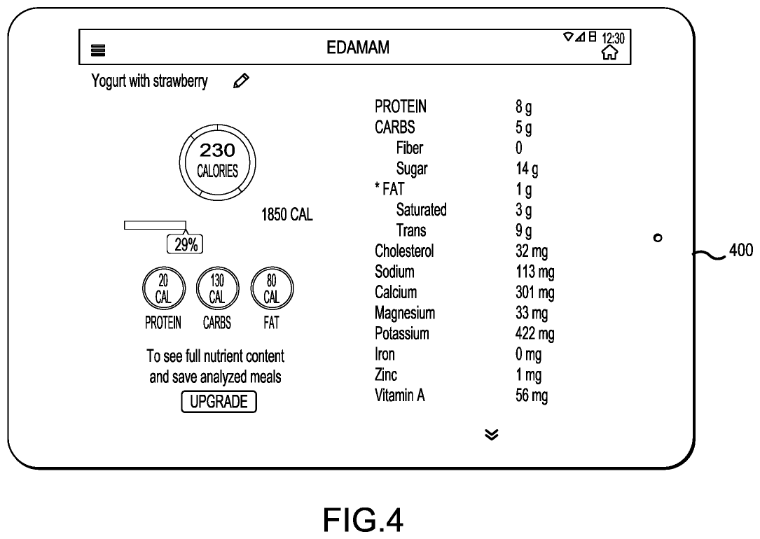

Finally, in step 324, the final step of process 300 the data is exported or deployed in a format that is usable to third parties such as companies using it in a machine format or humans that can access it through the nutrition wizard module 126 or a recipe search interface. The data is first indexed by any one or more of search instances module 108, API instances 110 and/or extractor module 116 in the above described process on search instances module 108, API instances module 110 or extractor module 116 for easier querying and the index containing unique identifier of each analyzed recipe, food or ingredient list, or combination thereof, is stored on elastic search cluster module 118. In an aspect of an embodiment of the present invention, the resultant data may be temporarily stored on search instances module 108, API instances module 110 and extractor module 116. In another aspect of an embodiment of the present invention, the data may be exported in one of the following ways: i) via API instances module 110 in a JSON format, with clearly defined fields for nutrients and diets; ii) via nutrition wizard module 126 in HTML format (as shown in FIG. 4) or exportable to PDF, Excel or CSV formats; iii) in other formats as needed by users, including export to CSV or text files.

Distillation:

An aspect of an embodiment of the present invention contemplates the development and provision of a generic representation of each data set in step 326 following storage of the data sets in step 322. In one instance, if database 120 has 200 representations for spaghetti Bolognese, extractor module 116 may, by execution of code, present a generic meal of spaghetti Bolognese by determining an average weighted representation having the most common ingredients, practices etc. amongst all 200 representations stored in database 120. Following this determination, the generic meal is stored in database 120.

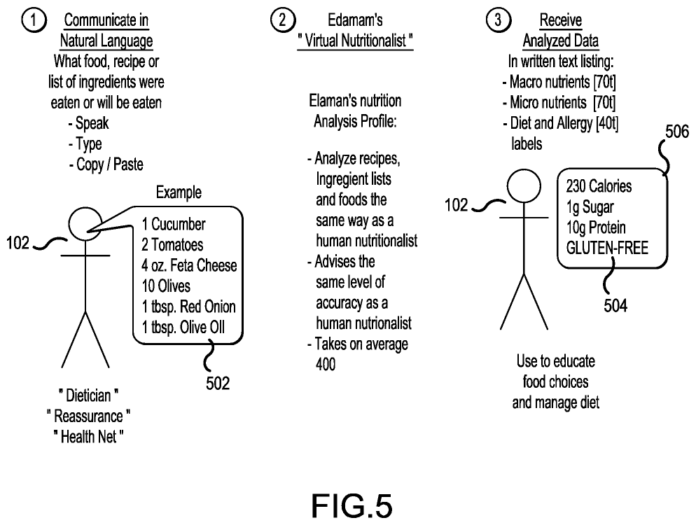

Referring now to FIG. 5, a user 102 is shown submitting information to system 100 via voice according to an aspect of an embodiment of the present invention. User 102 may use device 506 which may be enabled to use an intermediary or external voice recognition program such as Google Now, Amazon Alexa or Apple Ski. Upon provision of a voice command by user 102, search instances module 108, working with an intermediary or external voice recognition program, is enabled to receive the voice provided recipe 502. Search instances module 108, using applicable code, then transforms recipe 502 into text and is taken through process 300 as fully described above. Display of the extracted and analyzed recipe 504 is then provided to user 102 on device 506.

Aspects of embodiments of the present invention contemplate the use of modules in implementation of the process(es) outlined herein and operation of components of the system also disclosed herein. In an aspect of an embodiment of the present invention, the term “module” may represent self-contained computer hardware. In another aspect of an embodiment of the present invention, the term “module” may represent computer hardware on a designated computer chip or separate computer chips. In yet another aspect of an embodiment of the present invention, the processor may be configured to perform tasks not undertaken by the module(s) disclosed herein. In a further aspect of an embodiment of the present invention, the modules may be hardware resident on one chip, component, separate components, a remote server, database, some or each of which (or all of which, in one aspect of an embodiment of the present invention) may be separate and distinct from the device, or any combination thereof. In one aspect of an embodiment of the present invention, the processor may be configured to coordinate, implement and/or assign tasks to, from and/or among the module(s). In a preferred aspect of an embodiment of the present invention, “module” may represent operational cooperation between system components. For instance, an “extraction module” may comprise of the one or more processors, memory, computer executable instructions (code) executable by the one or more processors and resident within memory location(s) etc. with each component being in communication with one or more other components in the module and each component working with the other component(s) to conduct the desired operation for which the module is configured to do e.g. extraction of one or more data sets. In a further aspect of an embodiment of the present invention, module components may also, in an operational context, be components of other modules. In a preferred aspect of an embodiment of the present invention, “module” may include one or more processors, memory, computer executable instructions (code) executable by the one or more processors and resident within the memory.

In a further aspect of an embodiment of the present invention, operations or methods undertaken by system 100, and/or system 100's components or modules, as discussed above, may be implemented by execution, on one or more servers or processors configured to perform a predefined set of basic operations in response to receiving a corresponding basic instruction selected from a predefined native instruction set of codes. This native instruction set of codes or machine language instruction codes may be built into servers/processors of system 100's components. As such, different operations contemplated by the disclosure above, may be made possible by the selection of machine codes from specific machine language instruction codes. Additional complex operations may be made possible by the combination of different sets of machine language instruction codes.

Although this present invention has been disclosed with reference to specific forms and embodiments, it will be evident that a great number of variations may be made without departing from the spirit and scope of the present invention. For example, steps may be reversed, equivalent components or elements may be substituted for those specifically disclosed and certain features of the present invention may be used independently of other features—all without departing from the present invention as outlined above, in the appended drawings and the claims presented below.

What is claimed is:

1. A computer-implemented method of nutritional analysis comprising executing on at least one processor, the steps of:
enabling receipt of at least one collection of unstructured food related data, wherein the collection of unstructured food related data may be any one of at least one or more of: an ingredient, list of ingredients, a recipe, a food, a meal, or a combination of the forgoing;
extracting and identifying the at least one collection of unstructured food related data using a predetermined process, wherein extraction and identification of the at least one collection of unstructured data results in at least one structured data set;
mapping the at least one structured data set to an entity class;
combining the extracted the at least one structured data set into at least one construct;
combining the at least one construct into meaningful combinations of food, quantities, measures and techniques, wherein the combination results in at least one parsed combination; and
assigning nutrient content, diet information and allergen information to the at least one parsed combination resulting in final data, wherein the assignment is done using a database of nutrient content, diet information and allergen information for a wide range of foods.

2. The computer implemented method according to claim 1, wherein each step takes place by execution of specific sets of machine codes on at least one processor configured to perform a predefined set of basic operations in response to receiving a corresponding basic instruction selected from a predefined native instruction set of codes, wherein the specific sets of machine codes are selected from the native instruction set.

3. The computer implemented method according to claim 1 further comprising the step of applying a series of steps to make at least one adjustment to the at least one structured data set.

4. The computer implemented method according to claim 1 further comprising the additional steps of: checking for compatibility between the one or more constructs and ensuring that correct quantities and measures are applied to the at least one structured data set.

5. The computer implemented method according to claim 1 further comprising the step of adjusting sodium amount in the at least one structured data set.

6. The computer implemented method according to claim 1 further comprising the step of calculating fat absorption amount based on a frying technique parsed in the at least one structured data set.

7. The computer implemented method according to claim 1 further comprising any one or more steps of: calculating amount of nutrients in the final data, calculating recommended daily intake (RDI) for each nutrient, tagging the final data for a range of predefined diets.

8. A system for nutritional analysis comprising:
at least one input device;
one or more of: a search instances module, an API instances module, a scraper module for receiving at least one collection of unstructured food related data, wherein any one or more of the at least one input device, search instances module, API instances module, scraper module are configured for receipt of at least one collection of unstructured food related data wherein the at least one collection of unstructured food related data is any one or more of: an ingredient, a recipe, a food, a meal, a measure, a qualifier, a technique, a combination of the forgoing;
an extractor module, wherein the extractor module receives at least one collection of unstructured food related data from the scraper module and wherein any one or more of: the search instances module, API instances module, the extractor module operates upon the at least one collection of unstructured food related data; and
at least one database, in communication with the extractor module.

9. The system of claim 8, wherein the system further comprises of an elastic search cluster module in communication with any one or more of: the search instances module, the API instances module, the extractor module and the at least one database.

10. The system of claim 8 further comprising of a nutrition wizard interface module in communication with any one or more of: API instances module, extractor module, elastic search cluster modules, search instances module.

11. The system of claim 8, wherein the system further comprises of an entity loader module in communication with any one or more of: the search instances module, the API instances module, the extractor module.

12. A computer program product comprising a non-transitory computer readable medium having interfaces stored therein for causing a processor-based control logic to conduct nutritional analysis comprising executing on at least one processor, the steps of:
enabling receipt of at least one collection of unstructured food related data, wherein the collection of unstructured food related data may be any one of at least one or more of: an ingredient, a recipe, a food, a meal, a measure, a qualifier, a technique, a combination of the forgoing;
extracting and identifying the at least one collection of unstructured food related data using a predetermined process, wherein extraction and identification of the at least one collection of unstructured data results in at least one structured data set;
mapping the at least one structured data set to an entity class;
combining the extracted the at least one structured data set into at least one construct;
combining the at least one construct into meaningful combinations of food, quantities, measures and techniques, wherein the combination results in at least one parsed combination;
assigning nutrient content, diet information and allergen information to the at least one parsed combination resulting in final data, wherein the assignment is done using a database of nutrient content, diet information and allergen information for a wide range of foods.

13. The computer program product of claim 12 further comprising control logic for applying a series of steps to make at least one adjustment to at least one structured data set for a recipe as a whole.

14. The computer program product of claim 12 further comprising control logic for checking for compatibility between the one or more constructs and ensuring that correct quantities and measures are applied to the at least one structured data set.

15. The computer program product of claim 12 further comprising control logic for adjusting sodium amount in the at least one structured data set.

16. The computer program product of claim 12 further comprising control logic for calculating fat absorption amount based on a frying technique parsed in the at least one structured data set.

17. The computer program product of claim 16 further comprising computer readable codes for any one or more of: calculating amount of nutrients in the final data, calculating recommended daily intake (RDI) for each nutrient, tagging the final data for a range of predefined diets.

18. A non-transitory computer readable device having control logic stored therein for causing a computer-based interface to implement nutritional analysis, the control logic comprising computer readable program code for:
enabling receipt of at least one collection of unstructured food related data, wherein the collection of unstructured food related data may be any one of at least one or more of: an ingredient, a recipe, a food, a meal, a measure, a qualifier, a technique, a combination of the forgoing;
extracting and identifying the at least one collection of unstructured food related data using a predetermined process, wherein extraction and identification of the at least one collection of unstructured data results in at least one structured data set;
mapping the at least one structured data set to an entity class;
combining the extracted the at least one structured data set into at least one construct;
combining the at least one construct into meaningful combinations of food, quantities, measures and techniques, wherein the combination results in at least one parsed combination;
assigning nutrient content, diet information and allergen information to the at least one parsed combination resulting in final data, wherein the assignment is done using a database of nutrient content, diet information and allergen information for a wide range of foods.

19. The non-transitory computer readable device of claim 18, further comprising computer readable code for applying a series of steps to make at least one adjustment to any one or more of: an ingredient list or a recipe as a whole.

20. The non-transitory computer readable device of claim 18, further comprising computer readable code for checking for compatibility between the one or more constructs and ensuring that correct quantities and measures are applied to the at least one structured data set.

21. The non-transitory computer readable device of claim 18, further comprising computer readable code for adjusting sodium amount in the at least one structured data set.

22. The non-transitory computer readable device of claim 18, further comprising computer readable code for calculating fat absorption amount based on a frying technique parsed in the at least one structured data set.

23. The non-transitory computer readable device of claim 22 further comprising computer readable codes for any one or more of: calculating amount of nutrients in the final data, calculating recommended daily intake (RDI) for each nutrient, tagging the final data for a range of predefined diets.
